(19) United States
(12) Patent Application Publication
(10) Pub. No.: US 2014/0273155 A1
(43) Pub. Date: Sep. 18, 2014

(54) COMPOSITION AND METHOD FOR PRODUCING SAME

(71) Applicants: SUNSTAR ENGINEERING INC., Takatsuki-shi, Osaka (JP); SUNSTAR SINGAPORE PTE. LTD., Singapore (SG)

(72) Inventors: Haruka Miyao, Takatsuki-shi (JP); Yuzuru Ajima, Takatsuki-shi (JP); Toru Oka, Takatsuki-shi (JP); Denny Liauw, Singapore (SG)

(21) Appl. No.: 14/353,426

(22) PCT Filed: Oct. 25, 2012
(86) PCT No.: PCT/JP2012/077627

§ 371 Date: Apr. 22, 2014

(30) Foreign Application Priority Data
Oct. 28, 2011 (JP) 2011-238013

(51) Int. Cl. C12N 9/96

(52) U.S. Cl. CPC C12N 9/96
USPC 435/188

(57) ABSTRACT

To provide a novel and stable protein composition, and a method for stabilizing the protein composition. A composition that contains protein and water containing ultra-fine air bubbles having a mode particle size of 500 nm or less, and a method for stabilizing a protein composition that involves mixing protein and water containing ultra-fine air bubbles having a mode particle size of 500 nm or less.

### TECHNICAL FIELD

The present invention relates to a composition comprising a protein and water containing ultrafine bubbles having a mode particle size of no greater than 500 nm (such bubbles are also referred to as nanobubbles or NBs), as well as a method for stabilizing a protein composition which comprises mixing a protein with water containing ultrafine bubbles having a mode particle size of no greater than 500 nm.

### BACKGROUND ART

Proteins such as enzymes, antibodies and peptides are extensively used in detergents, industry, cosmetics, food processing, pharmaceuticals, diagnosis/examination, and biosensors. Water-soluble preparations/forms of proteins are easier to handle than powders and, because of this and other advantages, are commonly used in fields that involve the use of enzymes in large quantities. On the other hand, however, enzymes in aqueous solution are generally considerably instable and low in keeping quality as compared with the case where they are in powder form and, hence, are incapable of maintaining their physiological activity for an extended period of time. A conventionally known method for supplying proteins without lowering their physiological activity was to purify and stabilize the protein by freeze-drying or other means to avoid heat application and then supply the resulting preparation. Known methods for stabilizing proteins in aqueous solution include a process involving a step of incorporating polyhydric alcohols such as glycerin as stabilizers for uricase or peroxidase (Patent Document 1: JP Hei 6-70798A), a technique involving a step of adding bovine serum albumin or saccharides such as glucose or amino acids such as lysine to an aqueous solution containing cholesterol oxidase (Patent Document 2: JP Hei 8-187095A), and a technique in which an organic compound such as guanidine hydrochloride, urea or pyridine is added as a stabilizer to any kind of proteins in aqueous solution (Patent Document 3: JP 2011-67202A.)

These known techniques, however, have had several problems. Problems with the freeze-drying based method, for example, are that it is not applicable to proteins that will denature upon dehydration and that deterioration tends to result from moisture absorption and oxidation during the drying step, presenting the need to re-condition the aqueous solution of enzyme, which is simply a cumbersome procedure to adopt. The techniques of stabilizing proteins in aqueous solution are one of using polyhydric alcohols to stabilize uricase or peroxidase and another of using bovine serum albumin, saccharides or amino acids to stabilize an aqueous solution of cholesterol oxidase; but intending to stabilize only particular kinds of proteins, each of these methods has had the disadvantage of being limited in applicability. The method for using organic compounds such as guanidine hydrochloride to stabilize aqueous solutions of proteins has wide applicability but, on the other hand, it has the disadvantage of being inapplicable if the enzyme preparation formulated as a mixture containing a certain substance that reacts with the organic compound; as another problem, the concentration of the organic compound to be added must be adjusted to an appropriate value but again this is a cumbersome procedure to adopt.

### PRIOR ART LITERATURE

### Patent Literature

Patent Document 1: JP Hei 6-70798A

Patent Document 2: JP Hei 8-187095A

Patent Document 3: JP 2011-67202A

### SUMMARY OF INVENTION

### Problem to be Solved by the Invention

An object of the present invention is to provide novel, stable protein compositions that are free from the above-described problems. Another object of the present invention is to provide methods for stabilizing proteins without involving the above-described problems.

### Means for Solving the Problems

With a view to solving the above-described problems, the present inventors conducted intensive studies and found that stable protein compositions were obtained by mixing proteins with water containing ultrafine bubbles; the present invention has been accomplished on the basis of this finding.

The present invention provides a composition comprising a protein and water containing ultrafine bubbles having a mode particle size of no greater than 500 nm. The present invention also provides such a composition comprising a protein and water containing said ultrafine bubbles, wherein the mode particle concentration of ultrafine bubbles is at least 1×10⁶ bubbles per milliliter. The present invention further provides such a composition comprising a protein and water containing ultrafine bubbles, wherein the particle concentration of bubbles having a particle diameter of no greater than 1000 nm is at least 5×10⁷ bubbles per milliliter.

The present invention still further provides a method for stabilizing a protein composition which comprises mixing a protein with water containing ultrafine bubbles having a mode particle size of no greater than 500 nm. The present invention also provides a method for stabilizing a protein composition which comprises mixing a protein with water containing ultrafine bubbles having a mode particle size of no greater than 500 nm and in which the mode particle concentration of ultrafine bubbles is at least 1×10⁶ bubbles per milliliter. The present invention further provides a method for stabilizing a protein composition which comprises mixing a protein with water containing ultrafine bubbles having a mode particle size of no greater than 500 nm and in which the particle concentration of bubbles having a particle diameter of no greater than 1000 nm is at least 5×10⁷ bubbles per milliliter, provided that the mode particle concentration of ultrafine bubbles is optionally at least 1×10⁶ bubbles per milliliter.

In the present invention, the interior of the above-described ultrafine bubbles may be filled with one or more gases selected from a wide range of gases which include, but are not limited to, air, oxygen, hydrogen, nitrogen, carbon dioxide, argon, neon, xenon, fluorinated gases, and inert gases.

The proteins that can be used in the protein compositions of the present invention are not particularly limited and may include enzymes, animal-derived proteins, fish-derived proteins, plant-derived proteins, recombinant proteins, antibodies, peptides, etc. and the following may be given as examples that can be used.

Enzymes: Enzymes that can be used are oxidoreductases (e.g. cholesterol oxidase, glucose oxidase, ascorbate oxidase, polyphenol oxidase, and peroxidase); transferases (e.g. acyltransferase, sulfotransferase, and transglucosidase); hydrolases (e.g. protease, serine protease, amylase, lipase, cellulase, glucoamylase, and lysozyme); lyases (e.g. pectin lyase); isomerases (e.g. glucose isomerase); synthases (e.g. fatty acid synthase, phosphate synthase, citrate synthase, hyaluronate synthase, and carbonate dehydratase).

Recombinant proteins: Recombinant proteins that can be used are protein preparations (interferon a, growth hormone, insulin, and serum albumin), vaccines, etc.

Antibodies: Antibodies that can be used are monoclonal antibodies and polyclonal antibodies.

Peptides: Peptides that can be used are not limited to any particular amino acids and may include dipeptides, tripeptides, and polypeptides.

In a preferred mode of the present invention, the protein is a water-soluble protein; more preferably, the protein is an enzyme, and even more preferably it is a water-soluble enzyme; most preferably, the protein may be at least one enzyme selected from peroxidase, protease, cellulase, amylase, and lipase.

The amount of proteins to be used varies with factors such as their type and usage. Preferred amounts can be determined as appropriate by experiment and proteins can generally be used in the range from 1 ng/ml to 300 mg/ml, preferably from 10 ng/ml to 100 mg/ml, and more preferably from 30 ng/ml to 50 mg/ml.

The water to be used in the present invention may be selected from tap water, purified water, ion-exchanged water, pure water, ultrapure water, deionized water, distilled water, buffered water, clean water, natural water, filtered water, highly pure water, potable water, and electrolyzed water, but these are not the sole examples of water that can be used in the present invention.

If desired, water-soluble solvents may be added and examples include alcohols, glycols, glycerins, ethers, ketones, and esters.

### Advantageous Effects of the Invention

The protein containing compositions of the present invention are highly stable and, in particular, they exhibit high pH stability (being stable in the face of pH changes), high temperature stability (reducing the temperature effect), and high photostability (reducing the effect of light.)

### BRIEF DESCRIPTION OF THE DRAWINGS

FIG. 1 is a graph showing the result of measuring the particle size distribution of bubbles within water containing ultrafine bubbles.

FIG. 2 is a graph showing the result of measuring the particle size distribution of bubbles within purified water as specified in the Japanese Pharmacopoeia.

FIG. 3 is a graph showing the results of a test for measurement of catalase stability.

FIG. 4 is a graph showing the results of a test for measurement of lipase stability.

### MODES FOR CARRYING OUT THE INVENTION

The ultrafine bubbles to be used in the present invention have a mode particle size of no greater than 500 nm, preferably no greater than 300 nm, more preferably no greater than 150 nm, and most preferably no greater than100 nm, and the concentration of the bubbles with the mode particle size is preferably at least 1×10⁶, more preferably at least 3×10⁶, even more preferably at least 5×10⁶, still more preferably at least 7×10⁶, yet more preferably at least 1×10⁷, still more preferably at least 5×10⁷, even more preferably at least 9×10⁷, yet more preferably at least 1×10⁸, even more preferably at least 5×10⁸, and most preferably at least 9×10⁸ bubbles per milliliter.

In the present invention, the total particle concentration is preferably at least 5×10⁷, more preferably at least 7×10⁷, even more preferably at least 8×10⁷, still more preferably at least 1×10⁸, yet more preferably at least 6×10⁸, still more preferably at least 1×10⁹, even more preferably at least 3×10⁹, yet more preferably at least 5×10⁹, even more preferably at least 7×10⁹, still more preferably at least 1×10¹° , yet more preferably at least 2×10¹⁰, even more preferably at least 5×10¹⁰, and most preferably at least 7×10¹⁰ bubbles per milliliter. In a further preferred mode, bubbles larger than 1000 nm are virtually absent.

The particle diameter of the ultrafine bubbles to be used in the present invention is so small that it cannot be measured correctly with an ordinary particle size distribution analyzer. Hence, hereinafter, numerical values are employed that were obtained by measurements with the nanoparticle size analyzing system NanoSight Series (product of NanoSight Ltd.) The nanoparticle size analyzing system NanoSight Series (product of NanoSight Ltd.) measures the velocity of nanoparticles moving under Brownian motion and calculates the diameters of the particles from the measured velocity. A mode particle size can be verified from the size distribution of the particles present and represents the particle diameter for the case where the particles present at a maximum number.

It should be noted here that the particle diameter and number of ultrafine bubbles as referred to in the present invention are represented by numerical values as measured at the point in time when 24 hours have passed after the formulation of the composition.

It should also be noted that the composition of the present invention may contain additives including, but not limited to, antiseptics and stabilizers. Antiseptics that can be used include, but are not limited to, polyhexamethylene biguanide and paraben. Stabilizers that can be used include, but are not limited to, saccharides, antibiotics, aminoglycosides, organic acids, coenzymes, and amino acids. The composition of the present invention may also contain surfactants. Surfactants may be added as appropriate for use and other conditions, not only in the case where substances either insoluble or slightly soluble in water are contained as additives but also in the case of using water-soluble additives.

The zeta potential at surfaces of ultrafine bubbles have a certain effect on the stability of the bubbles. The surfaces of the ultrafine bubbles used in the present invention are electrically charged to provide a zeta potential of at least 5 mV, preferably at least 7 mV, more preferably at least 10 mV, even more preferably at least 20 mV, still more preferably at least 25 mV, and most preferably at least 30 mV, in absolute value. The absolute value of zeta potential is in proportion to the viscosity coefficient of a solution as divided by the dielectric constant of the solution, so the lower the temperature condition under which the water containing the ultrafine bubbles is mixed with a protein, the more likely it is that the bubbles have higher stability.

The ultrafine bubbles to be used in the present invention can be generated by any known means, such as the use of a static mixer, the use of a venturi tube, cavitation, vapor condensation, sonication, swirl formation, dissolution under pressure, or fine pore formation. A preferred method of bubble generation is by forming a gas-liquid mixture and shearing it.

An advantageous apparatus for generating ultrafine bubbles by the gas-liquid mix and shear method is disclosed in Japanese Patent No. 4118939. In this apparatus, the greater part of a gas-liquid mixture in fluid form introduced into a fluid swirling compartment does not simply flow toward the discharge port as in the apparatus already described in the Prior Art section but it first flows forming a swirl in the direction away from the discharge port. The swirl reaching the first end-wall member turns around and flows back toward the second end-wall member; since the returning swirl has a smaller radius of rotation than the swirl flowing toward the first end-wall member, it flows at a higher velocity, creating a sufficient shear force on the gas within the liquid to promote the formation of finer bubbles.

An aqueous solution of a protein is treated with a suitable apparatus to generate ultrafine bubbles in it, whereby the composition of the present invention can be produced that has the protein dissolved in the water. The composition of the present invention can also be produced by dissolving a protein in the water containing ultrafine bubbles. The water containing ultrafine bubbles may have the above-defined mode particle size and number of bubbles.

The foregoing description of the present invention and the description of the Examples that follow are only intended to provide a detailed explanation of various exemplary embodiments of the present invention and skilled artisans can make various improvements and changes of the embodiments disclosed herein without departing from the scope of the present invention. Therefore, the description herein will in no way limit the scope of the present invention, which shall be determined only by the recitation in the appended claims.

### EXAMPLES

### Preparation of Water Containing Atmospheric Ultrafine Bubbles

Ultrafine bubbles were generated in purified water (Japanese Pharmacopoeia) using BAVITAS of KYOWA KISETSU which was a device for generating ultrafine bubbles by the gas-liquid mix and shear method. The particle diameters of the generated ultrafine bubbles were measured with the nanoparticle size analyzing system NanoSight Series (product of NanoSight Ltd.) The result is shown in FIG. 1. The horizontal axis of the graph represents the particle diameter in nanometers and the vertical axis represents the number of NB particles (the number of nanobubble particles) per millimeter (10⁶/ml). FIG. 2 shows the result of a measurement of fine bubbles in the purified water of the Japanese Pharmacopoeia.

The water containing the generated ultrafine bubbles had a mode particle size of 86 nm; the particle concentration at the mode particle size was 7.57×10⁶ bubbles per milliliter and the total particle concentration was 6.86×10⁸ bubbles per milliliter.

The purified water of the Japanese Pharmacopoeia had such low particle concentrations that the size distribution was not normal distribution ; the result of the measurement was therefore attributed to noise.

In the following Examples, water containing atmospheric ultrafine bubbles was prepared by the same method as described above, and the blanks as

Comparative Examples used the purified water of the Japanese Pharmacopoeia in place of the water containing atmospheric ultrafine bubbles.

1. Stability of peroxidase at 20° C. or 50° C. for 10 days

To water containing atmospheric ultrafine bubbles, streptavidin peroxidase was added at a concentration of 50 ng/ml and the resulting mixture was dispensed into tubes in 1.0-ml portions; the tubes were then closed with an airtight stopper and stored at 20° C. or 50° C. for 1, 3 or 10 days. Subsequently, the stored aqueous solution of streptavidin peroxide was allowed to develop color by mixing 100 μl of the solution with 100 μl of a substrate solution prepared as described below.

### (Method for Preparing the Substrate Solution)

The reagents according to the recipe indicated below were mixed to prepare the substrate solution.

o-Phenylenediamine tablet (product of SIGMA-ALDRICH): one

70 mM citrate buffer (citric acid/sodium phosphate; pH 5.0): 12.5 ml

Hydrogen peroxide solution: 5 μl

### (Methods for Calculating the Concentration of Enzyme and the Percent Residual Enzyme Activity)

To the sample, 50 μl of a 4 N H₂SO₄ solution was added to quench the color reaction and thereafter the absorbance at 492 nm (A₄₉₂) was measured with a micro-plate reader (product of Thermo Fisher Scientific Inc.) The concentration of the enzyme was determined from a calibration curve constructed by using streptavidin peroxidase. The percent residual enzyme activity was calculated from the following equation, where A is the initial enzyme activity of the sample and B is the enzyme activity after storage:

Residual enzyme activity (%)=(1−(A−B)/A)×100

Note that the concentration at the mode particle size was calculated as a measured concentration value at the mode particle size (×10⁶ bubbles/ml) multiplied by the dilution ratio at measurement. The total particle concentration was calculated as a measured concentration value at the total particle concentration (×10⁸ bubbles/ml) multiplied by the dilution ratio at measurement.

The results are shown in the following Tables 1 and 2.

TABLE 1

| | | Comp. | Comp. | Comp. | | | |
|---|---|---|---|---|---|---|---|
| | | Ex. 1 | Ex. 2 | Ex. 2 | Ex. 1 | Ex. 2 | Ex. 3 |
| Test | Temperature of heat | 20 | 20 | 20 | 20 | 20 | 20 |
| conditions | treatment (° C.) | | | | | | |
| | Time of heat treatment (days) | 1 | 3 | 10 | 1 | 3 | 10 |
| | Mode particle size (nm) | — | — | — | 86 | 86 | 86 |
| | Concentration at mode | — | — | — | 7.57 | 7.57 | 7.57 |
| | particle size (×10⁶ bubbles/ml) | | | | | | |
| | Total particle concentration | — | — | — | 6.86 | 6.86 | 6.86 |
| | (×10⁸ bubbles/ml) | | | | | | |
| Enzyme | Residual enzyme (%) | 100.0 | 104.7 | 77.9 | 112.0 | 123.6 | 98.1 |
| activity | | | | | | | |

TABLE 2

| | | Comp. | Comp. | Comp. | | | |
|---|---|---|---|---|---|---|---|
| | | Ex. 4 | Ex. 5 | Ex. 6 | Ex. 4 | Ex. 5 | Ex. 6 |
| Test | Temperature of heat | 50 | 50 | 50 | 50 | 50 | 50 |
| conditions | treatment (° C.) | | | | | | |
| | Time of heat treatment (days) | 1 | 3 | 10 | 1 | 3 | 10 |
| | Mode particle size (nm) | — | — | — | 86 | 86 | 86 |
| | Concentration at mode | — | — | — | 7.57 | 7.57 | 7.57 |
| | particle size (×10⁶ bubbles/ml) | | | | | | |
| | Total particle concentration | — | — | — | 6.86 | 6.86 | 6.86 |
| | (×10⁸ bubbles/ml) | | | | | | |
| Enzyme | Residual enzyme (%) | 52.4 | 34.3 | 11.3 | 108.7 | 104.5 | 75.1 |
| activity | | | | | | | |

Examples 1 to 3 show the results in the case of storage at 20° C. for 1, 3, and 10 days. Examples 4 to 6 show the results in the case of storage at 50° C. for 1, 3, and 10 days. The comparison at 20° C. reveals marked improvements in percent residual enzyme. The results at 50° C. were also superb.

### 2. Stability of Enzyme to Heat (80° C.)

### (Preparation of Enzyme Solutions)

To water containing atmospheric ultrafine bubbles, streptavidin peroxidase was added at a concentration of 50 ng/ml and the resulting mixture was dispensed into tubes in 1.0-ml portions; the tubes were then closed with an airtight stopper and stored under the condition of 80° C. for 30 minutes. In the process, the mode particle concentration of the water containing atmospheric ultrafine bubbles was adjusted to an order of 10⁶ counts (Examples 7 and 8), an order of 10⁷ counts (Examples 9 and 10), and an order of 10⁸ counts (Examples 11 and 12), per milliliter.

Subsequently, the stored aqueous solution of streptavidin peroxide was allowed to develop color by mixing 100 μl of the solution with 100 μl of a substrate solution prepared from the reagents set out below.

### (Preparation of the Substrate Solution)

The reagents according to the recipe indicated below were mixed to prepare the substrate solution.

o-Phenylenediamine tablet (product of SIGMA-ALDRICH): one

70 mM citrate buffer (citric acid/sodium phosphate; pH 5.0): 12.5 ml

Hydrogen peroxide solution: 5

### (Methods for Calculating the Concentration of Enzyme and the Percent Residual Enzyme Activity)

To the sample, 50 μl of a 4 N H₂SO₄ solution was added to quench the color reaction and thereafter the absorbance at 492 nm was measured with a micro-plate reader (product of Thermo Fisher Scientific Inc.) The concentration of the enzyme was determined from a calibration curve constructed by using streptavidin peroxidase. The percent residual enzyme activity was calculated from the following equation, where A is the initial enzyme activity of the sample and B is the enzyme activity after storage:

Residual enzyme activity (%)=(1−(A−B)/A)×100
The results are shown in the following Table 3.
Obviously, the increased total particle concentration provided better results.

It is interesting to note that the residual enzyme was 0.0% in Example 8 and 15.2% in Example 10. Although the result of Example 10 was better than that of Comparative Example 8, the condition of 80° C. ×80 min is considered to require that the concentration at the mode particle size and the total particle concentration be desirably on the orders of 10⁸ and 10¹⁰, respectively, in terms of bubble counts as in Example 12.

TABLE 3

| | | | | | | Ex. 9 | Ex. 10 | Ex. 11 | Ex. 12 |
|---|---|---|---|---|---|---|---|---|---|
| | | | | | | (calculated | (calculated | (calculated | (calculated |
| | | Comp. | Comp. | | | particle con- | particle con- | particle con- | particle con- |
| | | Ex. 7 | Ex. 8 | Ex. 7 | Ex. 8 | centration) | centration) | centration) | centration) |
| Test | Temperature of heat | 80 | 80 | 80 | 80 | 80 | 80 | 80 | 80 |
| conditions | treatment (° C.) | | | | | | | | |
| | Time of heat treatment (min) | 30 | 80 | 30 | 80 | 30 | 80 | 30 | 80 |
| | Mode particle size (nm) | — | — | 79 | 79 | 76 | 76 | 76 | 76 |
| | Concentration at mode | — | — | 1.13 | 1.13 | 91.5 | 91.5 | 915 | 915 |
| | particle size (×10⁶ bubbles/ml) | | | | | | | | |
| | Total particle concentration | — | — | 0.89 | 0.89 | 70.8 | 70.8 | 708 | 708 |
| | (×10⁸ bubbles/ml) | | | | | | | | |
| Enzyme | Residual enzyme (%) | 0.73 | 0.0 | 24.7 | 0.0 | 76.2 | 15.2 | 95.5 | 57.6 |
| activity | | | | | | | | | |

### 3. Stability of Enzyme to pH

Using water containing atmospheric ultrafine bubbles, a citrate buffer (pH 4.6), an acetate buffer (pH 5.7), a phosphate buffer (pH 7.0), and a borate buffer (pH 8.9) were prepared; to each of these buffers, streptavidin peroxidase was added at a concentration of 50 ng/ml. The resulting mixtures were each dispensed into tubes in 1.0-ml portions; the tubes were then closed with an airtight stopper and stored under the condition of 80° C. for 20 minutes. Subsequently, the stored aqueous solution of streptavidin peroxide was allowed to develop color by mixing 100 μl of the solution with 100 μl of a substrate solution prepared from the reagents set out below.

### (Preparation of the Substrate Solution)

The reagents according to the recipe indicated below were mixed to prepare the substrate solution.

o-Phenylenediamine tablet (product of SIGMA-ALDRICH): one

70 mM citrate buffer (citric acid/sodium phosphate; pH 5.0): 12.5 ml

Hydrogen peroxide solution: 5 μl

### (Methods for Calculating the Concentration of Enzyme and the Percent Residual Enzyme Activity)

To the sample, 50 μl of a 4 N H₂SO₄ solution was added to quench the color reaction and thereafter the absorbance at 492 nm was measured with a micro-plate reader (product of Thermo Fisher Scientific Inc.) The concentration of the enzyme was determined from a calibration curve constructed by using streptavidin peroxidase. The percent residual enzyme activity was calculated from the following equation, where A is the initial enzyme activity of the sample and B is the enzyme activity after storage:

Residual enzyme activity (%)=(1−(A−B)/A)×100
The results are shown in the following Table 4.
Obviously, the present invention provided excellent stability over a wider pH range.

TABLE 4

| | | Comp. | Comp. | Comp. | Comp. | | | | |
|---|---|---|---|---|---|---|---|---|---|
| | | Ex. 9 | Ex. 10 | Ex. 11 | Ex. 12 | Ex. 13 | Ex. 14 | Ex. 15 | Ex. 16 |
| Test | Temperature of heat | 80 | 80 | 80 | 80 | 80 | 80 | 80 | 80 |
| conditions | treatment (° C.) | | | | | | | | |
| | Time of heat treatment (min) | 20 | 20 | 20 | 20 | 20 | 20 | 20 | 20 |
| | Buffer | Citrate | Acetate | Phosphate | Borate | Citrate | Acetate | Phosphate | Borate |
| | pH | 4.6 | 5.7 | 7.0 | 8.9 | 4.6 | 5.7 | 7.0 | 8.9 |
| | Mode particle size (nm) | — | — | — | — | 86 | 86 | 86 | 86 |
| | Concentration at mode | — | — | — | — | 7.57 | 7.57 | 7.57 | 7.57 |
| | particle size (×10⁶ bubbles/ml) | | | | | | | | |
| | Total particle concentration | — | — | — | — | 6.86 | 6.86 | 6.86 | 6.86 |
| | (×10⁸ bubbles/ml) | | | | | | | | |
| Enzyme | Residual enzyme (%) | 24.3 | 65.0 | 62.4 | 42.6 | 75.5 | 83.0 | 95.3 | 72.6 |
| activity | | | | | | | | | |

### 4. Heat (80° C.) Stability of Protease

To water containing atmospheric ultrafine bubbles, protease was added at a concentration of 10 mg/ml and after standing at 80° C. for 30 minutes, the resulting mixture was dispensed into tubes in 990-μl portions. After adding a solution of Azocasein Tris-Cl (1.3 M) and CaCl₂ (20 mM) in an amount of 10 μl to give a concentration of 0.05% (w/v), the tubes were placed on a heating block set at 60° C. (product of YAMATO SCIENTIFIC CO., LTD), closed with an airtight stopper, and then stored for 30 minutes.

Subsequently, 1.1 ml of 10% (w/v) trichloroacetic acid was added and the mixture was left to stand at room temperature (25-27° C.) for 30 minutes to quench the reaction. To separate the precipitating protein, the mixture was centrifuged at 13000×g for 10 minutes and the supernatant (1 ml) was allowed to develop color in a separate tube filled with 1 ml of 1 M NaOH.

### (Methods for Calculating the Enzyme Activity and the Percent Residual Enzyme Activity)

The prepared enzyme solution was subjected to a measurement of absorbance at 450 nm (A₄₅₀) with a micro-plate reader (product of Thermo Fisher Scientific Inc.) A blank was subjected to the same measurement of absorbance at 450 nm (A_{450b}). Using the two measurement values, enzyme activity was calculated from the following formula.

Enzyme activity (U/ml)=(A₄₅₀−A_{450b})/(0.001×30)

* One unit (U) of protease activity is defined as “an increase of 0.001 per minute in the absorbance at 450 nm.”

In the next step, the percent residual enzyme activity was calculated from the following equation, where A is the initial enzyme activity of the sample and B is the enzyme activity after 30-min storage:

Residual enzyme activity (%)=(1−(A−B)/A)×100
The results are shown in the following Table 5.
Obviously, the present invention also provided outstanding heat stability for protease.

TABLE 5

| | | | Ex. 17 |
|---|---|---|---|
| | | | (calculated |
| | | Comp. | particle |
| | | Ex. 13 | concentration) |
| Test | Temperature of heat | 80 | 80 |
| conditions | treatment (° C.) | | |
| | Time of heat treatment (min) | 30 | 30 |
| | Mode particle size (nm) | — | 99 |
| | Concentration at mode | — | 303 |
| | particle size (×10⁶ bubbles/ml) | | |
| | Total particle concentration | — | 215 |
| | (×10⁸ bubbles/ml) | | |
| Enzyme | Residual enzyme (%) | 24.8 | 65.0 |
| activity | | | |

### 5. Stability of Catalase

### (Testing Procedure)

(1) Catalase was dissolved in either purified water or water containing ultrafine bubbles (mode particle size, 100 nm; mode particle concentration, 8.77×10⁶/mL; total particle concentration at a particle diameter of no greater than 1000 nm, 6.18×10⁸/mL) to prepare an aqueous solution having a catalase concentration of 1 mg/mL (3809 Units/mL.)

(2) The aqueous catalase solution (1 mL) dispensed in microtubes was incubated in a thermo-shaker at 500 rpm and 62.5° C. for a predetermined period of time (20, 30, 40 or 50 min.) Thereafter, the aqueous solution was subjected to a treatment with a centrifuge (14000 rpm×5 min) to cause precipitation.

(3) A 100-μL portion of the supernatant of the thus treated aqueous catalase solution, 200 μL of 3% aqueous hydrogen peroxide, 200 μL of a phosphate buffer (500 mM, pH 5.8), and 100 μL of purified water were metered into tubes and subjected to reaction at room temperature for 15 minutes.

(4) After the reaction, heat treatment (100° C.×5 min) was conducted to achieve complete deactivation of the enzyme.

(5) The reaction mixture was metered in an amount of 100 μL and the absorbance at 290 nm was measured. From the measured values of absorbance, the percent decomposition of hydrogen peroxide was calculated using a calibration curve.

The results are shown in FIG. 3.

### 6. Stability of Lipase

### (Testing Procedure)

(1) Lipase was dissolved in either purified water or water containing ultrafine bubbles (mode particle size, 113 nm; mode particle concentration, 36.4×10⁶/mL; total particle concentration at a particle diameter of greater than 1000 nm, 20.9×10⁸/mL) to prepare an aqueous solution having a lipase concentration of 0.02 mg/mL (23.52 Units/mL.)

(2) The thus prepared aqueous lipase solution was incubated at 70° C. for 5 minutes.

(3) A 50-μL portion of the thus treated aqueous lipase solution, 100 of a substrate solution prepared by the method just described below, and 50 μL of a Tris-HCl buffer (pH 8.2) were subjected to reaction at room temperature for predetermined periods of time.

The reaction mixture was metered in a predetermined amount and the absorbance at 410 nm was measured.

The results are shown in FIG. 4.

### (Preparation of the Substrate Solution)

Purified water (100 mL) was mixed with 0.0135 g of p-nitrophenyl laurate, 0.017 g of sodium dodecyl sulfate, and 1.0 g of Triton X-100 and the added ingredients were dissolved in the water by heating the mixture at 65° C.; the solution was subsequently cooled.

1. A composition comprising a protein and water containing ultrafine bubbles having a
mode particle size of no greater than 500 nm.

2. The composition according to claim 1, wherein the mode particle concentration of ultrafine bubbles is at least 1×106 bubbles per milliliter.

3. The composition according to claim 1, wherein the particle concentration of bubbles having a particle diameter of no greater than 1000 nm is at least 5×107 bubbles per milliliter.

4. The composition according to claim 1, wherein the ultrafine bubbles are filled with one or more gases selected from air, oxygen, hydrogen, nitrogen, carbon dioxide, argon, neon, xenon, fluorinated gases, and inert gases.

5. The composition according to claim 1, wherein the protein is an enzyme.

6. The composition according to claim 5, wherein the enzyme is peroxidase, protease, cellulase, amylase, or lipase.

7. A method for stabilizing a protein composition which comprises mixing a protein with water containing ultrafine bubbles having a mode particle size of no greater than 500 nm.

8. A method for stabilizing a protein composition which comprises mixing a protein with water containing ultrafine bubbles having a mode particle size of no greater than 500 nm and wherein the mode particle concentration is at least 1×106 bubbles per milliliter.

9. The method according to claim 7, wherein the water containing ultrafine bubbles is such that the particle concentration of bubbles having a particle diameter of no greater than 1000 nm is at least 5×107 bubbles per milliliter.

10. The method according to claim 7, wherein the ultrafine bubbles include one or more gases selected from air, oxygen, hydrogen, nitrogen, carbon dioxide, argon, neon, xenon, fluorinated gases, and inert gases.

11. The method according to claim 7, wherein the protein is an enzyme.

12. The method according to claim 11, wherein the enzyme is peroxidase, protease, cellulase, amylase, or lipase.

13. The method according to claim 8, wherein the water containing ultrafine bubbles is such that the particle concentration of bubbles having a particle diameter of no greater than 1000 nm is at least 5×107 bubbles per milliliter.

14. The method according to claim 8, wherein the ultrafine bubbles include one or more gases selected from air, oxygen, hydrogen, nitrogen, carbon dioxide, argon, neon, xenon, fluorinated gases, and inert gases.

15. The method according to claim 8, wherein the protein is an enzyme.
